(19) United States
(12) Patent Application Publication
(10) Pub. No.: US 2013/0245622 A1
(43) Pub. Date: Sep. 19, 2013

(54) EXPANDABLE ELECTRODE DEVICE AND METHODS FOR NERVE MODULATION

(71) Applicant: BOSTON SCIENTIFIC SCIMED, INC., MAPLE GROVE, MN (US)

(72) Inventors: HUISUN WANG, MAPLE GROVE, MN (US); JAMES M. ANDERSON, FRIDLEY, MN (US)

(73) Assignee: BOSTON SCIENTIFIC SCIMED, INC., MAPLE GROVE, MN (US)

(21) Appl. No.: 13/802,349

(22) Filed: Mar. 13, 2013

Related U.S. Application Data
(60) Provisional application No. 61/612,626, filed on Mar. 19, 2012.

(51) Int. Cl. A61B 18/14

(52) U.S. Cl. CPC A61B 18/1492
USPC 606/41

(57) ABSTRACT

Embodiments of the disclosure provide an ablative system for nerve modulation through wall of a blood vessel. The ablative catheter system includes an elongate member having a proximal end and a distal end, a number of electrode elements, an expansion mechanism. The electrode elements are finger-like structures mounted at their proximal ends for pivotal rotation radially outward from the longitudinal axis of the elongate member from a collapsed state. Each electrode element having inner and an outer surface with an electrode portion connected to a source of electrical energy and an insulated portion and a slope surface forming the proximal portion of the inner surface, sloping outward from the longitudinal axis of the elongate member, and a tip portion at the distal portion of the inner surface, angled toward the longitudinal axis of the elongate member. The electrode element inner surfaces in the collapsed state define a central cavity.

### CROSS-REFERENCE TO RELATED APPLICATIONS

This application claims priority under 35 U.S.C. §119 to U.S. Provisional Application Ser. No. 61/612,626, filed Mar. 19, 2012, the entirety of which is incorporated herein by reference.

### TECHNICAL FIELD

The present disclosure relates to methods and apparatus for modulating nerves through the walls of blood vessels. More specifically, embodiments of the disclosure relate to ablation devices for intravascular nerve modulation.

### BACKGROUND

Certain treatments require temporary or permanent interruption or modification of select nerve functions. One example treatment is renal nerve ablation, which is sometimes used to treat conditions related to congestive heart failure. The kidneys produce a sympathetic response to congestive heart failure, which among other effects, increases the undesired retention of water and/or sodium. Ablating some nerves running to the kidneys may reduce or eliminate this sympathetic function, providing a corresponding reduction in the associated undesired symptoms. An ablation technique called rotoablation may be used for treatment of coronary heart disease by cleansing the coronary arteries. Rotablation consists of inserting a tiny, diamond-tipped, drill-like device into the affected artery to remove fatty deposits or plaque.

Many nerves (and nervous tissue such as brain tissue), including renal nerves, run along the walls of or in close proximity to blood vessels, and these nerves can be accessed intravascularly through the blood vessel walls. In some instances, it may be desirable to ablate perivascular renal nerves using a radio frequency (RF) electrode. Such treatment, however, may result in thermal injury to the vessel at the electrode and other undesirable side effects such as, but not limited to, blood damage, clotting, and/or protein fouling of the electrode. To prevent such undesirable side effects, some techniques attempt to increase the distance between the vessel walls and the electrode.

Therefore, there remains a need for ablation devices and methods for intravascular nerve modulation which may provide a uniform contact between the electrodes and the vessel wall.

### SUMMARY

The disclosure is directed to several alternative designs, materials, and methods of manufacturing medical device structures and assemblies.

Accordingly, some embodiments pertain to an ablative catheter system for nerve modulation through wall of a blood vessel. The ablative catheter system includes an elongate member having a proximal end and a distal end, a number of electrode elements, an expansion mechanism. The electrode elements are finger-like structures mounted at their proximal ends for pivotal rotation radially outward from the longitudinal axis of the elongate member from a collapsed state. Each electrode element having inner and an outer surface with an electrode portion connected to a source of electrical energy and an insulated portion and a slope surface forming the proximal portion of the inner surface, sloping outward from the longitudinal axis of the elongate member, and a tip portion at the distal portion of the inner surface, angled toward the longitudinal axis of the elongate member. The electrode element inner surfaces in the collapsed state define a central cavity.

Some other embodiments pertain to a method for ablating a nerve perivascularly through a blood vessel. The method includes advancing an ablative catheter system intravascularly proximate a desired location in a blood vessel. The ablative catheter system includes an elongate member having a number of electrode elements extending from its distal end, with each electrode member having an outer surface carrying one or more electrode portions and one or more insulated portions, and an actuator mounted on the distal end of a pull wire extending longitudinally through the elongate, the electrode elements being radially expandable between a collapsed state and an expanded state. The method further includes deploying the actuator to expand the electrode elements to the expanded state, wherein portions of the insulated portions uniformly contact the walls of the blood vessel and the plurality of electrode elements are at a distance from the walls of the blood vessel. Next, electricity may be conducted through the electrode elements to ablate a portion of the blood vessel.

An example system for nerve modulation may include an elongate member having a proximal end and a distal end. A plurality of electrode elements may extend from the distal end. The electrode elements may be finger-like structures mounted at their proximal ends for pivotal rotation radially outward from the longitudinal axis of the elongate member from a collapsed state. Each electrode element may have an inner and an outer surface and an electrode portion connected to a source of electrical energy on the outer surface. The system may also include an actuation mechanism for moving the plurality of electrode elements from a collapsed configuration to an expanded configuration.

An example method for ablating a renal nerve may include providing a catheter system. The catheter system may include an elongate member having a proximal end and a distal end. A plurality of electrode elements may extend from the distal end. The electrode elements may be finger-like structures mounted at their proximal ends for pivotal rotation radially outward from the longitudinal axis of the elongate member from a collapsed state. Each electrode element may have an inner and an outer surface and an electrode portion connected to a source of electrical energy on the outer surface. The system may also include an actuation mechanism for moving the plurality of electrode elements from a collapsed configuration to an expanded configuration. The method may also include advancing the system through a body lumen to a position within a renal artery, deploying the actuation mechanism to expand the electrode elements to the expanded state, and energizing at least some of the electrode elements.

Also disclosed are medical devices. An example medical device may include an elongate catheter shaft having a distal portion. A plurality of electrode fingers may be coupled to the distal portion and may extend distally therefrom. The electrode fingers may be configured to shift between a first configuration and an expanded configuration. Each of the electrode fingers may include an electrode region. Each of the electrode fingers may also include a sloped inner surface. The medical device may also include an actuation member for shifting the electrode fingers between the first configuration and the expanded configuration. The actuation member may include an engagement member that is configured to engage the sloped inner surface of each of the electrode fingers.

The summary of some example embodiments is not intended to describe each disclosed embodiment or every implementation of the disclosure.

### BRIEF DESCRIPTION OF THE DRAWINGS

The present disclosure may be more completely understood in consideration of the following detailed description of various embodiments in connection with the accompanying drawings, in which:

FIGS. 1A-1B are schematic views illustrating an exemplary ablative catheter system including an expansion actuation pull member, in accordance with an embodiment of the present disclosure;

FIG. 2 illustrates the ablative catheter system in expanded state, in accordance with an embodiment of the present disclosure;

FIG. 3 is a schematic view illustrating an exemplary ablative catheter system including a sheath, in accordance with another embodiment of the present disclosure;

FIG. 4A illustrates a cross sectional view of an exemplary ablative catheter system in a collapsed position;

FIG. 4B illustrates a cross sectional view of an exemplary ablative catheter system in an expanded position; and

While embodiments of the present disclosure are amenable to various modifications and alternative forms, specifics thereof have been shown by way of example in the drawings and will be described in detail. It should be understood, however, that the intention is not to limit aspects of the disclosure to the particular embodiments described. One the contrary, the intention is to cover all modifications, equivalents, and alternatives falling within the spirit and scope of the present disclosure.

### DETAILED DESCRIPTION

For the following defined terms, these definitions shall be applied, unless a different definition is provided in the claims or elsewhere in the specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the term “about” may be indicative as including numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

Although some suitable dimension ranges and/or values pertaining to various components, features, and/or specifications are disclosed, one of skill in the art, incited by the present disclosure, would understand desired dimensions, ranges and/or values many deviate from those expressly disclosed.

As used in this specification and the appended claims, the singular forms “a,” “an,” and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The detailed description and the drawings, which are not necessarily to scale, depict illustrative embodiments and are not intended to limit the scope of the disclosure. The illustrative embodiments depicted are intended only as exemplary. Selected features of any illustrative embodiment may be incorporated into an additional embodiment unless clearly stated to the contrary.

While the devices and methods described herein are discussed relative to renal nerve modulation, it is contemplated that the devices and methods may be used in other applications where nerve modulation and/or ablation are desired.

In some instances, it may be desirable to ablate perivascular renal nerves with deep target tissue heating. The energy passes from an electrode to the desired treatment region to heat the tissue. The circulating fluid in the vessel (e.g. blood) may cool the wall of the vessel while allowing therapeutic temperatures to be achieved in the deep tissue.

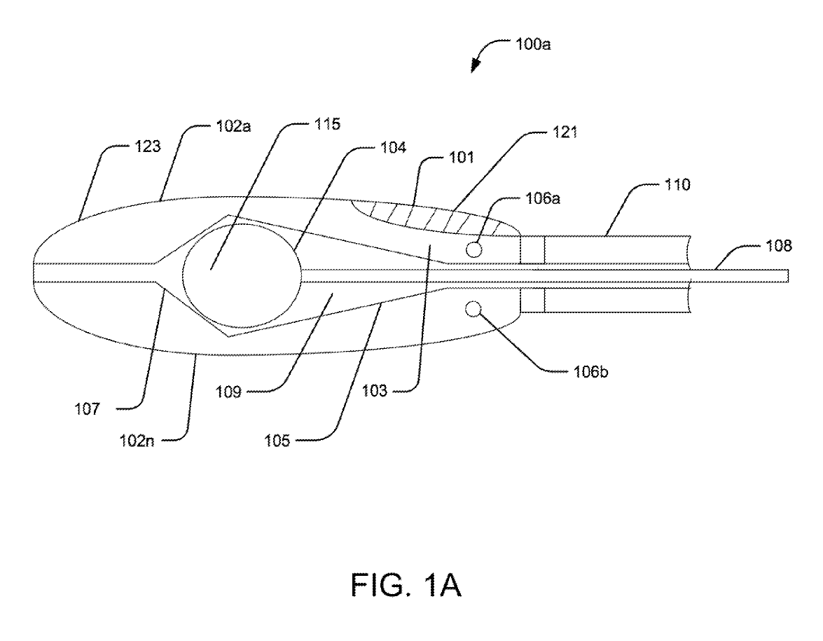

FIGS. 1A-1B are schematic views illustrating an exemplary ablative catheter system 100a, 100b. Here, the term “ablative catheter system 100” may be used to collectively refer the ablative catheter system 100a or 100b or both.

As shown in FIG. 1A, the ablative catheter system 100a may include an elongate member 110 having a proximal end and a distal end. The elongate member 110 (or the proximal tube) can be a conventional catheter, generally but not necessarily terminating in a proximal tube. Also, the ablative catheter system 100a may include a number of electrode elements 102a-n extending distally from the distal end of the elongate member.

Each electrode element, individually referred to by reference numeral 102, may be a finger-like structure connected to the proximal end of the elongate member 110. Each electrode element is pivotally carried on a hinge or pin member 106a-b, allowing the member to rotate radially outward from the longitudinal axis of the elongate member 110. Further, each electrode element has an outer surface 101 and an inner surface 103, respectively oriented away from and toward the longitudinal axis of the elongate member.

The inner surface 103 of electrode elements 102a-n are shaped to collectively define a central or inner cavity 109. From the proximal end of each electrode element, a sloped surface 105 is angled away from the longitudinal axis of elongate member 110. Toward the distal end of the electrode element, a tip portion 107 angles back toward the longitudinal axis. When lying mutually parallel, as shown in FIG. 1A, sloped surfaces 105 and tip portions 107 leave a volume enclosed by the electrode elements, forming central cavity 109.

The number and particular form and dimensions of electrode elements 102a-n are chosen to fit a particular therapeutic situation or scenario. In an embodiment of the present disclosure, the ablative catheter system 100a may include at least three electrode elements. In another embodiment, the multiple electrode elements 102a-n may be present at an outer surface of the elongate member of the ablative catheter system 100a.

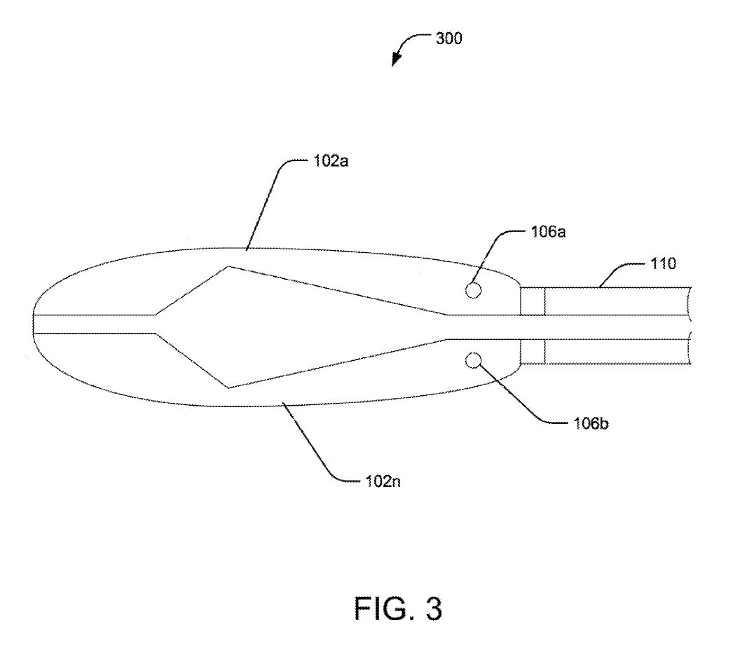

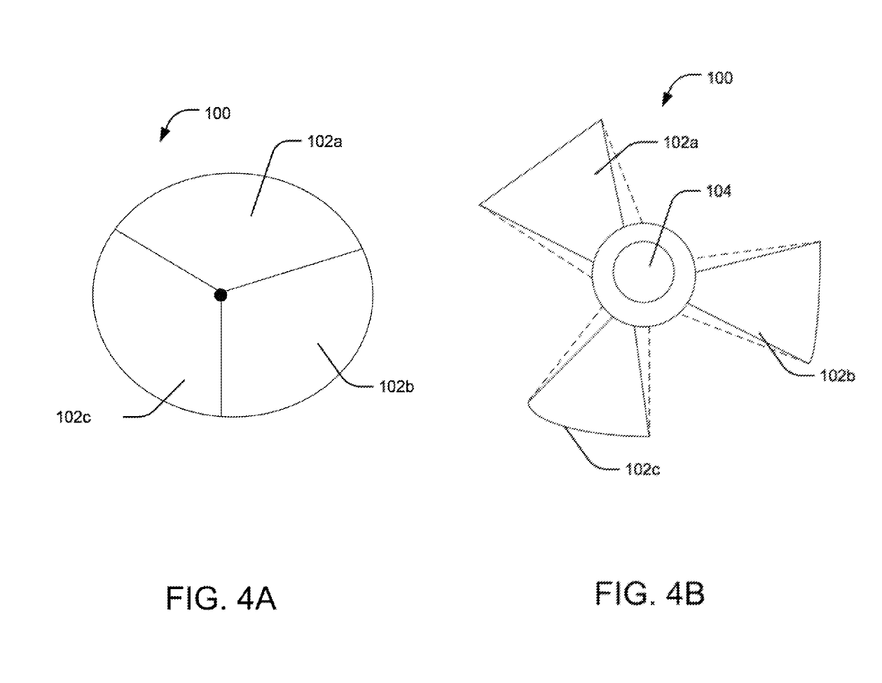

An expansion mechanism 115 serves to move the electrode elements 102a-n between a collapsed state, shown in FIG. 1A, and an expanded state shown in FIG. 4B. The expansion mechanism includes a pull wire 108, which extends longitudinally all the way through the elongate member 110, terminating at its proximal end and a control mechanism, such as a handle. This wire can be a conventional control wire, well known in the art. Actuator 104 is mounted on the distal end of pull wire 108, attached by a durable attachment means, such as welding, brazing, or the like. In the illustrated embodiment, actuator 104 takes the form of a sphere, but those in the art will recognize that a number of suitable shapes can be chosen for this element.

As shown in FIG. 1A, actuator 104 is dimensioned to fit within central cavity 109. The particular dimensions may be chosen to provide a snug fit or a relatively loose fit, at the discretion of the designer. As will be clear from the discussion below, it may be seen to be advantageous to provide a relatively snug fit for actuator 104.

Expansion mechanism 115 impels electrode elements 102a-n from a collapsed to an expanded state. That movement occurs when an operator causes pull wire 108 to move proximally, which in turn moves actuator 104 in a proximal direction as well. The actuator makes contact with the sloped surface 105 of inner surface 103, and the interaction between the spherical actuator and the sloped surface impels each electrode element 102 to rotate radially outward away from the longitudinal axis of the elongate member 110. This allows the electrode elements to be positioned up against the wall of the vessel. The axial symmetric nature of the expansion may allow each electrode element 102 to be positioned against the wall with an amount of force substantially equal to that of the other electrode elements.

As can easily be seen, the collapsed state of the ablative catheter system 100 can be useful for advancing the system to a therapeutic site, such as a renal blood vessel having a perivascular nerve that is the target of ablation treatment. When the system is positioned adjacent the target treatment area or surface, electrode elements 102a-n are converted to the expanded state, bringing electrode portions 123 into position adjacent vessel walls, where electrical energy can be applied to ablate target tissue.

The outer surface 101 of each of the electrode elements 102a-n includes both insulated portions 121 and electrode portions 123. As the name implies, insulated portions 121 are defined by an electrically insulating material, such as a polymer or other known insulator, and the electrode portions 123 can transmit electrical current. Those portions could be, for example, bare metal. Insulated and electrode portions are arranged in a pre-defined pattern. For example, electrode elements 102a-n may be staggered by altering the pre-defined pattern of the insulation sections to provide staggered electrode contact areas. A staggered pattern for electrode portions 123, for example, may be useful for avoiding ablation in a complete circumferential ring of a blood vessel. As can easily be understood, ablation in a complete ring could lead to an area of weakness in the blood vessel. Moreover, altering the position of insulated portions 121 to the distal portions of electrode element 102 could allow ablation to proceed in an off-wall fashion.

In alternative embodiments of the present disclosure, hinges 106 a-b could contain spring elements to bias the electrode elements 102a-n toward either a collapsed or expanded state. If biased to a collapsed state, actuator 104 would work against the biasing force when pull wire 108 was moved proximally. In systems where the electrode elements are biased toward the expanded state, a conventional sheath element can be used to control the position of the electrode elements.

In one embodiment, shown in FIG. 1B, mechanical hinges 106 are replaced by a resilient non-conductive element 121 such as, rubber, certain polymers, or any equivalent elastic, resilient material.

Though not shown, the ablative catheter system 100a (or 100b) includes one or more conductors for providing power to the electrode elements 102a-n. A proximal end of each conductor may be connected to a control and power element, which supplies the necessary electrical energy to activate the one or more electrode elements 102a-n at or near a distal end. The control and power element may include monitoring elements to monitor parameters such as power, temperature, voltage, pulse size and/or shape and other suitable parameters as well as suitable controls for performing the desired procedure. In some instances, the power element may control a radio frequency (RF) electrode. The RF electrode may be configured to operate at a frequency of approximately 460 kHz. It is contemplated that any desired frequency in the RF range may be used, for example, from 450-500 kHz. However, it is contemplated that different types of energy outside the RF spectrum may be used as desired, for example, but not limited to ultrasound, microwave, and laser energy.

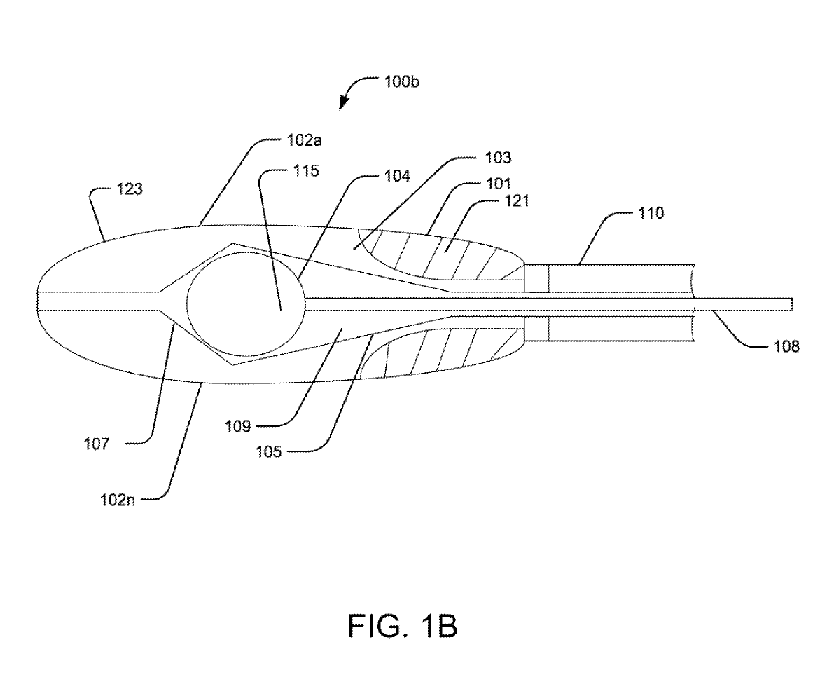

FIG. 2 illustrates a side view of the ablative catheter system 100 in an expanded state, in accordance with an embodiment of the present disclosure. There, it can be seen that actuator 104 has moved in a proximal direction, as shown by arrow A. In the course of that movement, actuator 104 applied force to the sloped surface 105 of inner surface 103. That force caused the electrode elements 102a-n to rotate radially, pivoting on hinges 106 a-b. Consequently, the distal end of each electrode element 102 rotates toward the walls of a treatment site blood vessel in preparation for the application of electrical energy in the ablation process.

In expanded position, the electrode elements 102a-n may touch the walls of the blood vessel uniformly. Further, the position of the electrode elements 102a-n can be shifted to vary the longitudinal location of the electrodes so that they do not form an ablated ring of tissue in the blood vessel. The ablative catheter system 100 can be inserted or advanced in the patient's body in a closed position. Thereafter, the ablative catheter system 100 can be deployed at a desired location or place within the patient's body for example, coronary artery, by retracting the actuator 104 to force the electrode elements 102a-n apart. Electricity may be conducted to the electrode elements 102a-n through the actuator 104 to activate the ablative catheter system 100 and to ablate a selective portion of the blood vessel. In an embodiment, the electricity may be conducted to the electrode elements through the elongate member 110 to which electrode elements 102a-n are connected.

In the expanded state, some portions of the electrode elements 102a-n may contact the blood vessel walls and some portions remain at a distance from the walls of the blood vessels. Electrodes elements 102a-n may be positioned on the portions of the elongate member that remain at a distance from the wall. Depending on the desired application, the electrode elements 102a-n may be placed along the actuator 104. For example, in one instance, electrode elements 102a-n may be placed slightly away from the wall. Alternatively, electrode elements 102a-n may be placed such that they are centered in the blood vessel.

In some embodiments, various actuation elements, including self-actuation elements, can replace the structure illustrated here. For example, inflating means (not shown) may be employed, including balloons inflated by fluids, or dilators. Other such inflating means may also be utilized without departing from the scope of the present disclosure. For example, means such as springs, or levers may be utilized to expand the actuator 104. Similarly, the actuator 104, itself, may be formed of pivotal structures connected to one another. For instance, the member may be formed of multiple wires connected to one another along pivotal joints. An outward force on the pivotal point expands the various wires connected to the point, expanding the actuator 104.

The expansion of the actuator 104 should be such that it does not cause damage to the artery or the blood vessels by exerting a large force on the vessel walls. To prevent such large expansion diameters, the actuator 104 may include visualization devices such as cameras or fluorescent dyes to visualize the extent of expansion. Further, the actuator 104 may include a force or expansion-limiting component that prevents the member from expanding beyond a certain limit. Often, the expansion limit may be set during manufacturing of the member. For example, operators may know the average size of renal arteries, and they may ensure the cage does not expand beyond the average artery size. For example, the diameter of the ablative catheter system 100 may be maintained below about 4 French.

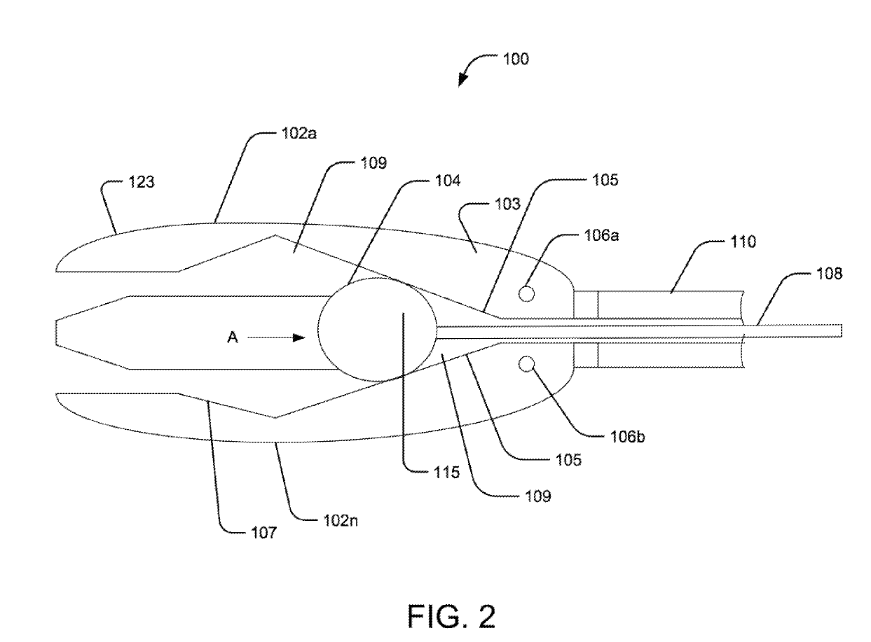

FIG. 3 is a schematic view illustrating an exemplary ablative catheter system 300, in accordance with another embodiment of the present disclosure. As described with reference to FIG. 1, the ablative catheter system 100 may include a number of electrode elements 102a-n, the pull wire 108, the elongate member 110, any one of the mechanical hinges 106a-n or the non-conductive resilient element 121, and the actuator 104. The ablative catheter system 300 may include the electrode elements 102a-n, the elongate member 110, any one of the mechanical hinges 106a-b or the non-conductive resilient element 121 (not illustrated). In ablative catheter system 300, the hinges 106a-b or a resilient element biases the electrode elements 102a-n towards the expanded state. The electrode elements 102a-n are joined to each other at the proximal end by the mechanical hinges 106a-b, which hinge biases the electrode elements 102a-n towards the expanded state. Retraction of a sheath (not illustrated) allows the system 300 to deploy and expand the electrode elements 102a-n towards the wall.

The elongate member 110 of the ablative catheter system 300 extends along the elongate axis from the proximal end of the ablative catheter system 300. The connection to the elongate member 110 may be temporary or permanent. Examples of temporary connection include snap-fit, Luer-lock, or screw-fit. Examples of permanent connection include welding or gluing. It will be understood that various other connection means are known in the art and any of these means may be incorporated to connect the various members. In other instances, the elongate member 110 including a number of electrode elements 102a-n may not be connected to the elongate member 110. Using an independent elongate member including a number of electrode elements 102a-n and the sheath 302 may allow operators to use the ablative catheter system 300 for other procedures or to insert guidewires for guiding and urging the catheter to the desired location.

In one embodiment, the elongate member includes conductor covered by insulation portions in a pre-defined pattern which can be altered. In an embodiment, the pre-defined pattern of the insulation section may be altered depending on the desired application of the ablative catheter system 300. The proximal end of the conductor may be connected to a power source such as an external power generator or battery incorporated in the elongate member 110. In an embodiment, the ablative catheter system 100 or 300 may include three electrodes

FIG. 4A illustrates a cross sectional view of an exemplary ablative catheter system 100 in a collapsed position. As described with reference to FIGS. 1A-1B, the ablative catheter system 100 may include an elongate member, a number of electrode elements 102a-n, the actuator 104, the pull wire 108, and the elongate member 110. In the illustrated embodiment, the ablative catheter system 100 may include three electrode elements. Further, the ablative catheter system 100 can be advanced within a blood vessel in a closed position, either. After reaching a desired position, the ablative catheter system 100 can be deployed and expanded. The actuator 104 may be formed of a conductor with an insulation section. Portions of the electrode elements 102a-n that contact the vessel surfaces may include the insulation section, and portions where electrode elements 102a-n are present may be bare. For example, the center portion of the electrode elements 102a-n may be without an insulation section, while all other portions may have the insulation. When electrical signals are passed through the conductor, the bare portions may emanate these signals behaving as the electrode elements 102a-n. Therefore, based on the required number and position of electrode elements 102a-n, portions of the elongate member may be left bare.

FIG. 4B illustrates a cross sectional view of an exemplary ablative catheter system in an expanded position. From this state, the ablative catheter system 100 may be expanded using numerous techniques depending on the properties of the ablative catheter system 100. For instance, the electrode elements of the ablative catheter system 100 may be self-expandable or expanded by some external force. Self-expandable members may be formed of any material that is in a compressed state when force is applied and in an expanded state when force is released. Such members may be formed of shape memory alloys such as Nitinol or any other self-expandable material commonly known in the art.

Many techniques may be utilized to apply a force on a self-expandable member and to hold it in the compressed state. According to one technique, the ablative catheter system 100 is present within the sheath 302 as shown in FIG. 3 for deployment. The sheath 302 may exert a radially inward pressure on the electrode elements 102a-n keeping it in the compressed state. Once the electrode elements 102a-n exits the sheath 302, however, the pressure is released, and the electrode elements 102a-n expands. In an embodiment, the ablative catheter system 100 or 300 may include three electrodes. It will be understood that in such situations, the material and thickness of the sheath 302 is selected such that it applies a greater force on the electrode elements 102a-n than the force exerted by the electrode elements 102a-n on the sheath 302. If the sheath 302 material is too thin or too elastic, it may not be sufficient to hold the electrode elements 102a-n in the compressed state and the electrode elements 102a-n may expand within the sheath 302 itself. Alternatively, if the sheath 302 is too rigid or thick, it may not be able to traverse the circuitous vasculature path, causing injury to the vessel walls. Therefore, it may be often preferred to select a suitable material and thickness keeping both aspects in mind.

According to another technique, the pull wire 108 may be utilized to expand the electrode elements. The pull wire 108 may be attached to the elongate member's distal end or proximal end. When the pull wire 108 is pulled in a certain axial direction (distally or proximally), it places a tensile force on the actuator 104, stretching it longitudinally and keeping it in the compressed state. When the pull wire 108 is released, the tensile force is released permitting the actuator 104 to enter the expanded state. For example, if the pull wire 108 is attached to the member's distal end, pulling the wire distally elongates (compresses) the actuator 104 and releasing the pull wire 108, releases the force on the actuator 104, expanding it. Moreover, means to pull, push, or release the pull wire 108 may be configured in the proximal tube or at handle of the elongate member of the ablative catheter system 100 or 300 allowing operators to easily expand or compress the actuator 104, as required. Alternatively, the actuation means may be present at the proximal end of the elongate member 110.

The ablative catheter system 100 (or 300) can be intravascularly advanced proximate to a desired location in a blood vessel. Then, the actuator 104 can be deployed in an expanded position in the blood vessel such that portions of the insulation sections uniformly contact the walls of the blood vessel and the electrode elements are at a distance from the walls of the blood vessel. The electrode elements 102a-n is then activated to ablate nerve tissue. During this procedure, the ablative member may continuously monitor the temperature at the electrode elements 102a-n and the vessel walls. Further, the electrode elements 102a-n may be activated sequentially or simultaneously, as desired. Known radiography techniques may be utilized to monitor the tissue being ablated. Once the tissue is sufficiently ablated, the catheter system may be advanced or the ablative member may be retracted to compress the ablative member and retrieve it from the patient's body.

Those skilled in the art will recognize that the present disclosure may be manifested in a variety of forms other than the specific embodiments described and contemplated herein. Accordingly, departure in form a and detail may be made without departing from the scope and spirit of the present disclosure as described in the appended claims.

What is claimed is:

1. A system for nerve modulation, comprising:
an elongate member having a proximal end and a distal end;
a plurality of electrode elements extending from the distal end, the electrode elements being finger-like structures mounted at their proximal ends for pivotal rotation radially outward from the longitudinal axis of the elongate member from a collapsed state;
wherein each electrode element has an inner and an outer surface, with an electrode portion connected to a source of electrical energy on the outer surface; and
an actuation mechanism for moving the plurality of electrode elements from a collapsed configuration to an expanded configuration.

2. The system of claim 1, wherein each electrode element has a sloped surface forming a proximal portion of the inner surface such that the plurality of electrode elements define a cavity and wherein the actuation mechanism comprises a pull wire and an enlarged element on a distal end of the pull wire and disposed in the cavity and configured such that proximal movement of the pull wire forces the enlarged element against the sloped surface of each of the electrode elements to pivotably rotate the electrode elements.

3. The system of claim 2, wherein the enlarged element is a sphere.

4. The system of claim 1, wherein the actuation mechanism comprises a wire extending distally from the plurality of electrode elements and operably connected to the plurality of electrode elements such that relative longitudinal movement between the wire and the elongate member operates to move to the plurality of electrode elements to the expanded configuration.

5. The system of claim 1, wherein the plurality of electrode elements are biased to the expanded configuration and wherein the actuation mechanism comprises a sheath disposed around the plurality of electrode elements and slidable with respect thereto.

6. The system of claim 1, wherein each of the electrode elements comprises an insulated portion.

7. The system of claim 6, wherein the insulated portion of each electrode member is proximal the electrode portion.

8. The system of claim 1, wherein the electrode elements define a central cavity when in the collapsed configuration and wherein the actuation mechanism is disposed within the central cavity.

9. The system of claim 1, wherein the plurality of electrode elements are pivotally mounted at their proximal ends by a mechanical hinge.

10. The system of claim 9, wherein the mechanical hinge biases the electrode elements toward the collapsed configuration.

11. The system of claim 1, wherein the plurality of electrode elements are joined to each other at the proximal end by a non-conductive resilient element.

12. The system of claim 11, wherein the resilient element biases the plurality of electrode elements toward the collapsed state.

13. The system of claim 1, wherein the actuation mechanism is configured to conduct electricity to the plurality of the electrode elements.

14. The system of claim 1, wherein the location of each of the electrode portions of the plurality of electrode elements are staggered longitudinally.

15. A method for ablating a renal nerve, the method comprising:
providing a catheter system, the catheter system comprising:
an elongate member having a proximal end and a distal end,
a plurality of electrode elements extending from the distal end, the electrode elements being finger-like structures mounted at their proximal ends for pivotal rotation radially outward from the longitudinal axis of the elongate member from a collapsed state to an expanded state,
wherein each electrode element has an inner and an outer surface, with an electrode portion connected to a source of electrical energy on the outer surface, and
an actuation mechanism for moving the plurality of electrode elements from a collapsed configuration to an expanded configuration;
advancing the system through a body lumen to a position within a renal artery;
deploying the actuation mechanism to expand the electrode elements to the expanded state; and
energizing at least some of the electrode elements.

16. The method of claim 15, wherein advancing the system through a body lumen to a position within a renal artery includes contacting a wall of the renal artery with the electrode portions.

17. The method of claim 15, wherein each of the electrode elements comprises an insulated portion.

18. The method of claim 17, wherein deploying the actuation mechanism to expand the electrode elements to the expanded configuration includes positioning the insulated portions against a wall of the renal artery.

19. The method of claim 18, wherein deploying the actuation mechanism to expand the electrode elements to the expanded state includes spacing the electrode portions from the wall of the renal artery.

20. A medical device, comprising:
an elongate catheter shaft having a distal portion;
a plurality of electrode fingers coupled to the distal portion and extending distally therefrom, the electrode fingers being configured to shift between a first configuration and an expanded configuration;
wherein each of the electrode fingers includes an electrode region;
wherein each of the electrode fingers includes a sloped inner surface;
an actuation member for shifting the electrode fingers between the first configuration and the expanded configuration; and
wherein the actuation member includes an engagement member that is configured to engage the sloped inner surface of each of the electrode fingers.
